(19) United States
(12) Patent Application Publication
(10) Pub. No.: US 2004/0010261 A1
(43) Pub. Date: Jan. 15, 2004

(54) Tool for releasably gripping an orthopedic implant

(76) Inventors: Stephen H. Hoag, Warsaw, IN (US); Justin S. Hertzler, Warsaw, IN (US); Robert L. Brubaker, North Manchester, IN (US); David M. Blakemore, Warsaw, IN (US)

Correspondence Address: BAKER & DANIELS, 111 E. WAYNE STREET, SUITE 800, FORT WAYNE, IN, 46802

(21) Appl. No.: 10/194,744

(22) Filed: Jul. 12, 2002

(51) Int. Cl. A61F 2/00

(52) U.S. Cl. 606/99

(57) ABSTRACT

A tool for releasably gripping an orthopedic implant. The tool includes a first gripping member which may be a threaded shaft and a second gripping member which may be the tool body. The first gripping member has a gripping end engageable with the implant and secures the implant to the tool with a bearing surface located on the distal end of the tool engaging the implant. The distal end may also include a feature to rotationally engage the implant and resist relative rotational movement between the tool and implant. A release mechanism is provided which, when the implant is secured to the tool, is operable to release the gripping members from their relative positions and retract the bearing surface from the implant. The first gripping member which then only loosely engages the implant may be completely disengaged from the implant. The release mechanism may be a pivoting lever body.

### BACKGROUND OF THE INVENTION

1. Field of the Invention

The present invention relates to instrumentation for use with orthopedic implants and, more particularly, to a tool for gripping and inserting an orthopedic implant such as a femoral stem prosthesis.

2. Description of the Related Art

A femoral stem prosthesis, and other long bone prostheses, generally include an elongated stem which is inserted into the intramedullary canal of the long bone after the canal has been prepared to receive the prosthesis. The prosthesis must be gripped to properly insert and position the stem of the prosthesis in the prepared canal. Relative movement between the tool used to grip the prosthesis and the prosthesis is undesirable during the insertion procedure since such relative motion could have a negative impact on the proper positioning of the prosthesis within the canal. It is also undesirable for the tool to grip the prosthesis in a manner that might scratch or otherwise damage the prosthesis and potentially render the prosthesis unusable. Once the prosthesis has been properly positioned within the bone, the gripping tool must be released. During the release of the gripping tool from the prosthesis it is undesirable for the releasing action to impart any relative motion between the prosthesis and the bone in which the prosthesis has been properly positioned. For such prostheses which are cemented within the intramedullary canal, releasing the prosthesis without imparting any relative movement between the implant and the surrounding cement mantle and thereby avoid disturbing the bond between the implant and cement is of particular importance.

### SUMMARY OF THE INVENTION

The present invention provides a tool for releasably gripping an orthopedic prosthesis or implant which enables the tool to firmly grip the implant in a manner which is not likely to damage the implant and also provides for releasing the tool from the implant after its insertion in a bone in a manner which is unlikely to disturb the proper positioning of the implant relative to the bone.

The invention comprises, in one form thereof, a tool for releasably gripping an orthopedic implant having a recess with an internal surface wherein the tool includes a first gripping member having a gripping end. The gripping end is insertable into the implant recess and engageable with internal surface to prevent removal of the gripping end from the recess by non-rotational, linear translational movement of the gripping end relative to the implant. The tool also includes a second gripping member having a bearing surface wherein, with the gripping end engaged with the internal surface of the implant recess, the second gripping member is translatable along a first axis relative to the first gripping member between an engaged position wherein the bearing surface is bearingly engaged with the implant proximate the recess and a disengaged position wherein the bearing surface is spaced from the implant. The tool also includes a non-threaded release mechanism operable to axially release the second gripping member from its engaged position.

The tool may include a first gripping member which has an elongate shaft and a radially projecting protrusion and a second gripping member which has a sleeve defining a bore. The shaft is rotatably disposed in said bore and the gripping end is rotationally engageable with the internal surface. The gripping end extends outwardly from one end of the bore and the protrusion is at least partially exposed at an opposite end of the bore. The protrusion limits the extent to which the shaft can be translated within the bore towards the end at which the gripping end extends and may also provide a site for the manual grasping of the shaft and thereby facilitate the rotation of the shaft.

The tool may also include a release mechanism which includes a lever body pivotable about a lever axis which is disposed substantially perpendicular to the first direction in which the two gripping members are relatively translatable and moves the second gripping member between its engaged and disengaged positions. The first gripping member may also include an elongate shaft and the second member include a sleeve defining a bore. The shaft is relatively moveably disposed within the bore with a distal end of the sleeve defining an opening to the bore. The gripping end extends outwardly from the opening and the bearing surface is disposed on the distal end proximate the opening. The gripping end may also be rotationally engageable with the internal surface and the first gripping member may further include an at least partially exposed, radially projecting protrusion disposed at an end of the shaft opposite the gripping end.

The invention comprises, in another form thereof, a tool for releasably gripping an orthopedic implant having a threaded cavity wherein the tool includes a first gripping member having a threaded end threadingly engageable with the threaded cavity by relative rotation of the first gripping member and the implant about a first axis. The first gripping member is postionable in at least one gripping position relative to the implant wherein the threaded end is threadingly engaged with the threaded cavity and is rotatable in both directions from the gripping position about the first axis relative to the implant. The tool also includes a second gripping member having a bearing surface wherein, with the first gripping member in the gripping position, the second gripping member is translatable relative to the first gripping member in a direction substantially parallel to the first axis between an engaged position wherein the bearing surface is bearingly engaged with the implant proximate the threaded cavity and a disengaged position wherein the bearing surface is spaced from the implant. The tool also includes a release mechanism which is non-rotatable about the first axis and which can release the first gripping member from a fixed axial position relative to the second gripping member when the second gripping member is in the engaged position.

The invention comprises, in yet another form thereof, a tool for releasably gripping an orthopedic implant having an internally threaded cavity wherein the tool includes a first gripping member having a shaft with a threaded end engageable with the cavity. The tool also includes a tool body having a sleeve defining a bore extending through the sleeve. One end of the sleeve defines a distal end of the tool. The distal end defines an opening to the bore and a bearing surface proximate the opening. The shaft is rotatably disposed within the bore and extends through the opening to expose at least a portion of the threaded end. The tool also includes a lever body pivotable about a lever axis. The lever axis is oriented substantially perpendicular to the first axis. The lever body is engageable with the first gripping member and the tool body. Pivotal motion of the lever body axially displaces the first gripping member relative to the tool body with the lever body being pivotally moveable between a first lever position wherein the threaded end extends a first distance outwardly from the sleeve opening to a second lever position wherein the threaded end extends a second distance outwardly from the sleeve opening. The first distance is greater than the second distance. The lever body is operable to release the first gripping member from an axial position relative to the tool body defined by the lever body being in the second lever position with the threaded end and bearing surface being engaged with the implant.

The tool may also have a first gripping member which includes an at least partially exposed, radially projecting protrusion disposed on an end of the shaft opposite the threaded end. The lever body is positioned between the threaded end and the protrusion and is engageable with the protrusion.

The tool may also include a distal end having a feature which rotationally engages the implant to thereby resist relative rotational movement of the tool and the implant.

The tool may also have a release mechanism which includes a biased engagement member. The engagement member is biased into a position interlocking the lever body and the tool body when the lever body is positioned in the second lever position.

An advantage of the present invention is that it provides a tool which firmly grips the implant in a manner which is not likely to damage the implant.

Another advantage of the present invention is that it provides a tool which can be released from the implant, after the insertion of the implant in a bone, in a manner which is unlikely to disturb the proper positioning of the implant relative to the bone.

Yet another advantage of the present invention is that it provides, in some embodiments thereof, a gripping tool which releasably grips an orthopedic implant by the threaded engagement of the implant with a first gripping member and the axial engagement of the implant by a second gripping member. The tool is released from the implant after its insertion in a bone by the axial disengagement of the second gripping member followed by the rotational disengagement of the threaded gripping member. This manner of releasing the tool from the implant facilitates the maintenance of the proper positioning of the implant relative to the bone during the release of the tool from the implant.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a perspective view of a tool in accordance with the present invention.

FIG. 2 is a top view of the tool.

FIG. 3 is an exploded side view of the tool and an orthopedic implant.

FIG. 4 is an end view of the tool.

FIG. 5 is a top view of the tool body of the tool of FIG. 1.

FIG. 6 is a side view of the tool body.

FIG. 7 is an end view of the tool body.

FIG. 8 is a side view of the lever body of the tool of FIG. 1.

FIG. 9 is a bottom view of the lever body.

FIG. 10 is an end view of the lever body.

FIG. 11 is a cross sectional view of the lever body taken along line 11-11 of FIG. 8.

FIG. 12 is a schematic illustration of a ball plunger which may be used with the tool.

Corresponding reference characters indicate corresponding parts throughout the several views. Although the exemplification set out herein illustrates an embodiment of the invention, in one form, the embodiment disclosed below is not intended to be exhaustive or to be construed as limiting the scope of the invention to the precise form disclosed.

### DESCRIPTION OF THE PRESENT INVENTION

A tool 20 for releasably gripping an orthopedic implant 22 such as a femoral stem prosthesis is shown in the drawings. Femoral stem prostheses are well known in the art and typically include an elongate stem adapted for insertion into the prepared intermedullary canal of a femur, a neck and a head. Implant 22 shown in FIG. 3 is a femoral stem prosthesis which utilizes a modular head (not shown).

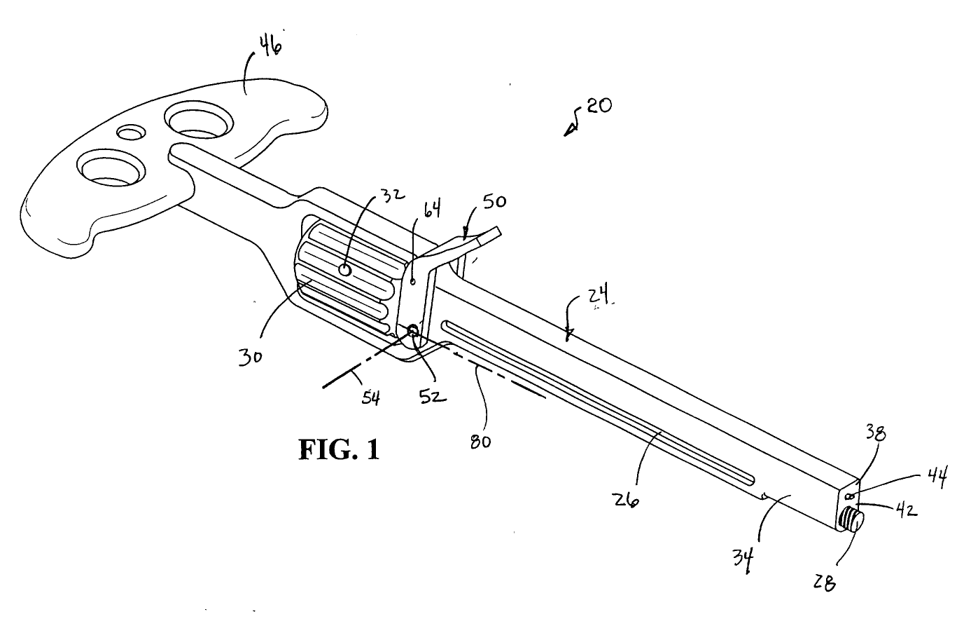

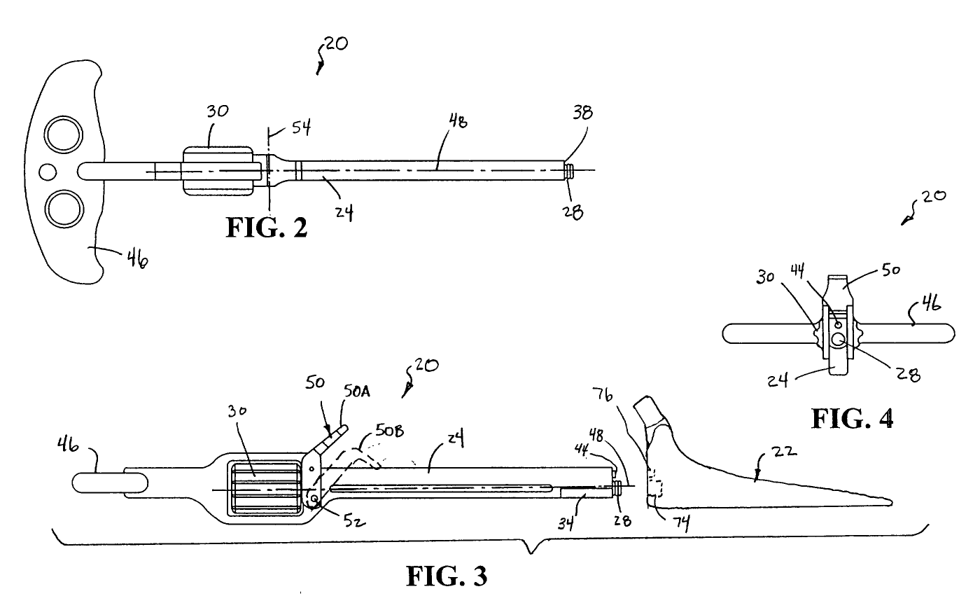

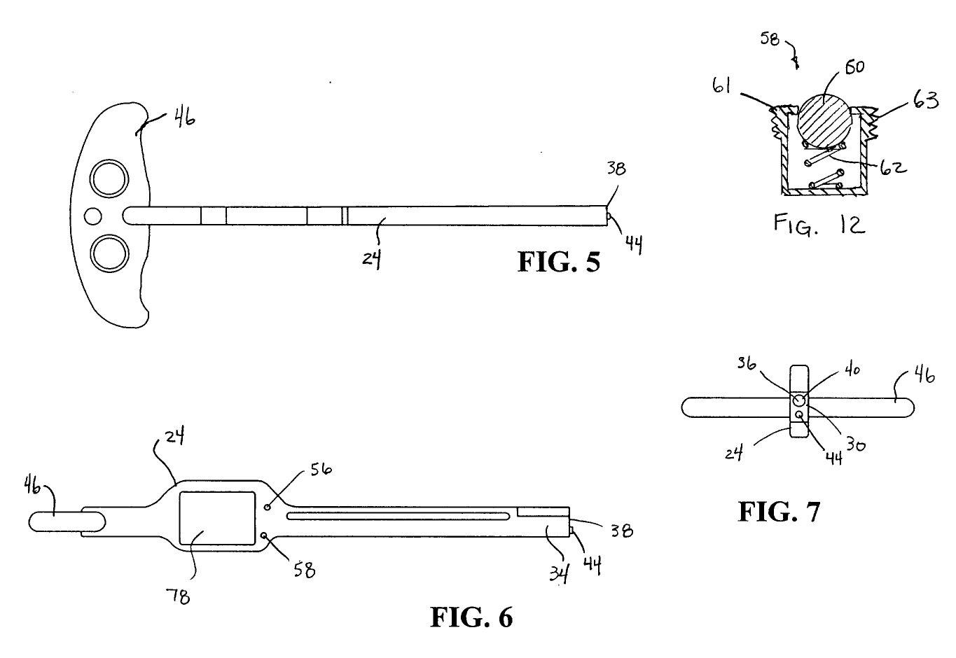

As can be seen in FIG. 1, tool 20 includes a tool body 24 and a first gripping member 26. Tool body 24 forms a second gripping member for securing implant 22 as explained in greater detail below. The first gripping member 26 takes the form of an elongated shaft with a gripping end 28 formed by a threaded end in the illustrated embodiment. A radially projecting protrusion, or knob, 30 is located opposite threaded end 28 and is secured to shaft 26 by a pin 32. The tool body 24 includes a sleeve 34 which defines a bore 36. Shaft 26 is positioned in bore 36 with threaded end 28 extending outwardly from distal end 38 of sleeve 34 which defines an opening 40 to bore 36. Shaft 26 can rotate within bore 36. Distal end 38 defines a bearing surface 42 and includes a projection 44 as discussed in greater detail below. Tool body 24 also includes a handle 46 located opposite distal end 38 and oriented transverse to axis 48 defined by shaft 26 and bore 36.

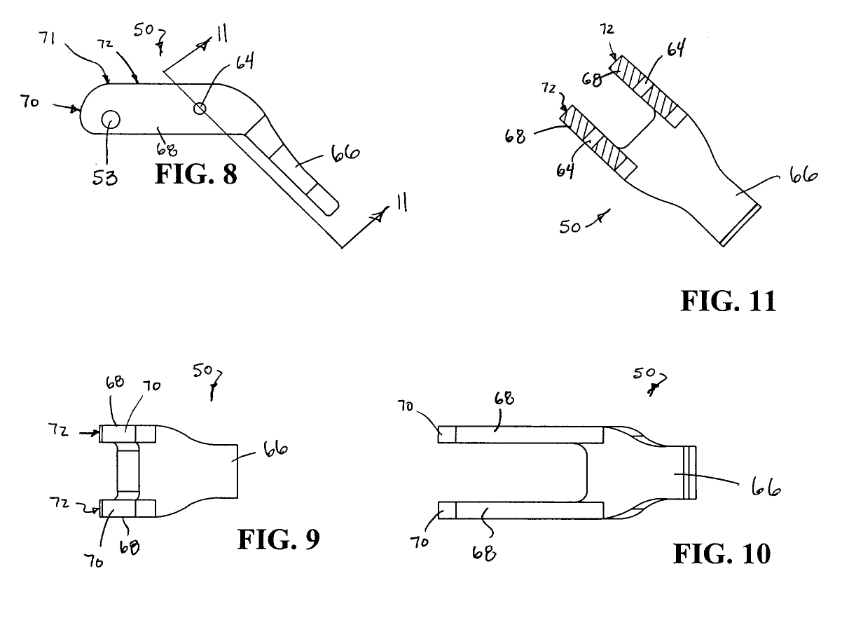

Tool 20 can be manufactured out of stainless steel or other suitable materials using conventional manufacturing techniques. As shown in FIG. 1, a lever body 50 is attached to tool body 24. Lever body 50 is pivotally attached to tool body 24 by pivot pin 52 which extends through opening 53 in lever body 50. Pivot pin 52 defines a lever axis 54 which is oriented substantially transverse to axis 48 and about which lever body 50 is pivotally moveable. Pivot pin 52 is mounted in opening 56 in tool body 24. Tool body 24 also includes a ball plunger mechanism 58 which includes an engagement member 60 biasing member 62 located in housing 61. In the illustrated embodiment engagement member 60 is a sphere and biasing member 62 is a spring as schematically illustrated in FIG. 12. Housing 61 is secured in opening 56 with external threads 63. Sphere 60 is received in opening 64 in lever body 50 when lever body 50 is in the position shown in FIG. 1. As can be seen with reference to FIGS. 8-11, lever body 50 includes a grip 66 and two spaced apart lever arms 68. The lever arms 68 each define a camming surface 70 and a securement surface 72 which abut each other at the location indicated by reference number 71 shown in FIG. 8.

The illustrated implant 22 includes a threaded recess 74 and a second recess 76. Implant 22 is secured to tool 20 by first placing lever body 50 in the position 50A shown in solid lines in FIG. 3 in which threaded end 28 extends outwardly from distal end 38. Lever body 50 is held in this engaged position 50A relative to tool body 20 by mechanism 58 which engages both tool body 24 at opening 56 and lever body 50 at opening 64 to thereby interlock lever body 50 and tool body 24 together in this position. In the illustrated embodiment, a mechanism 58 is located on only one side of tool body 24. Mechanism 58 is engaged and disengaged by grasping grip 66 and manually moving lever body 50. Mechanism 58 provides sufficient interconnection of lever body 50 and tool body 24 to inhibit the inadvertent disengagement of lever body 50 and tool body 24 but is not designed to transfer significant forces between these two bodies such as those forces transferred by shaft 26 by the attachment of an implant 22.

After placing lever body 50 in position 50A, gripping end 28 is then engaged with the internal surface of recess 74. Gripping end 28 is engaged with recess 74 in a manner which prevents the removal of gripping end 28 from recess 76 by non-rotational, linear translation of gripping end 28 relative to implant 22 to thereby allow gripping end 28 to hold implant 22 in tight engagement against bearing surface 42. The use of a gripping end 28 which rotationally engages the internal surface of recess 74 can provide such an engagement. In the illustrated embodiment, gripping end 28 is rotated relative to tool body 24 and implant 22 into threading engagement with threaded recess 74.

A portion of knob 30 is exposed at opening 78 in tool body 24 and can be manually grasped to impart rotational motion to shaft 26. Projection 44 is seated in recess 76 as implant 22 is engaged by threaded end 28. Shaft 26 is rotated until threaded end 28 firmly secures implant 22 to tool 20. In its engaged position, bearing surface 42 which is disposed proximate opening 40 bears against implant 22 and thereby resists relative rotational motion between implant 22 and tool 20. Bearing surface 42 can also impart axial forces to implant 22 during the insertion of implant 22 into the intramedullary canal of a bone. Although bearing surface 42 may resist relative rotational movement of implant 22 and tool 20, in the illustrated embodiment it is the engagement of projection 44 and recess 76 that provides the primary means by which such relative rotational motion is prevented. In addition to projection 44, alternative features for rotationally engaging distal end 38 and implant 22 to resist relative rotational movement between tool 20 and implant 22 could be employed. For example, a recess could be located on distal end 38 and a projection on the implant 22.

When the implant is attached to tool 20, shaft 26 is placed in tension as knob 30 bears against securement surfaces 72 on lever body 50 and threaded end 28 is engaged with recess 76 which causes implant 22 to bear against bearing surface 42. Securement surface 72 is oriented substantially transverse to axis 48 and positioned to intersect with a line 80 parallel to axis 48 which passes through lever axis 54 when lever body 50 is in position 50A to facilitate the transfer of forces from the shaft 26 to tool body 24. In the illustrated embodiment, such a line passes through securement surface 72 proximate the abutment 71 of securement surface 72 and camming surface 70.

To disengage the implant 22 from tool 20 after implant 22 has been inserted into the intramedullary canal of a femur, lever body 50 is first pivoted to its disengaged position 50B shown in dashed outline in FIG. 3. In this disengaged position, threaded end 28 extends a greater distance outwardly from distal end 38 than when lever body 50 is placed in its engaged position 50A discussed above. As explained in greater detail below, lever body 50 forms a release mechanism which, when an implant 22 is secured to tool 20, allows for the relative axial release of tool body 24 and shaft 26. After this initial axial release, implant 22 is still engaged with tool 20 but is not tightly secured thereto. The complete disengagement of implant 22 from tool 20 is accomplished by the unthreading of gripping end 28 from recess 74.

The outward extension of threaded end 28 is accomplished by pivotal movement of lever body 50 from position 50A to position 50B. As shown in FIG. 3, lever body 50 is positioned between radially projecting protrusion, or knob, 30 and threaded end 28. When lever body 50 is in position 50A, securement surface 72 engages knob 30 which forms a part of gripping member 26. During pivotal movement of lever body 50 from position 50A to position 50B and in position 50B, camming surface 70 engages knob 30. The smallest distance between securement surface 72 and lever axis 54 is where line 80 intersects securement surface 72 which is proximate abutment location 71. Camming surface 70 defines an arcuate surface separated from lever axis 54 by a variable distance which is no greater than this distance between securement surface 72 and pivot axis 54 along line 80 with that portion of camming surface 70 having the greatest separation being proximate abutment location 71. This separation distance between camming surface 70 and lever axis 54 progressively decreases as camming surface 70 extends away from abutment location 71. This configuration of securement surface 72 and camming surface 70 results in the displacement of shaft 26 relative to tool body 24 along axis 48 as lever body 50 is pivoted from position 50A to position 50B with knob 30 moving increasingly closer to lever axis 54 as lever body 50 is pivoted from position 50A to position 50B.

By axially translating shaft 26 in bore 36 to outwardly extend threaded end 28, implant 22 is disengaged from its tight fit against bearing surface 42 in a manner which does not impart rotational forces to implant 22 which might disturb the positioning of implant 22 in the femur in which it has been inserted. After outwardly extending threaded end 28, threaded end 28 can be rotated within threaded recess in either direction since bearing surface 42 is spaced from implant 22. In this loosely threaded condition, threaded end 28 can be rotated relative to implant 22 to complete the disengagement of threaded end 28, and hence tool 20, from implant 22 without imparting significant torque to implant 22. Thus, by having a non-threaded release mechanism, i.e., lever body 50, which, with implant 22 tightly secured to tool 20, does not rotate about axis 48 to initially release first gripping member, or shaft, 26 and second gripping member, or tool body, 24 from their relative axial positions, tool 20 can be disengaged from implant 22 in a manner unlikely to disturb the positioning of implant 22.

While this invention has been described as having an exemplary design, the present invention may be further modified within the spirit and scope of this disclosure. This application is therefore intended to cover any variations, uses, or adaptations of the invention using its general principles.

What is claimed is:

1. A tool for releasably gripping an orthopedic implant having a recess with an internal surface, said tool comprising:
a first gripping member having a gripping end, said gripping end being insertable into the recess, said gripping end being engageable with the internal surface to prevent removal of said gripping end from the recess by non-rotational, linear translation of said gripping end relative to the implant;
a second gripping member having a bearing surface wherein, with said gripping end engaged with the internal surface, said second gripping member is translatable relative to said first gripping member along a first axis between an engaged position wherein said bearing surface is bearingly engaged with the implant proximate the recess and a disengaged position wherein said bearing surface is spaced from the implant; and
a non-threaded release mechanism operable to axially release said second gripping member from said engaged position.

2. The tool of claim 1 wherein said first gripping member comprises an elongate shaft and a radially projecting protrusion and said second gripping member comprises a sleeve defining a bore, said shaft being rotatably disposed in said bore, said gripping end being rotationally engageable with the internal surface and extending outwardly from one end of said bore and said protrusion being at least partially exposed at an opposite end of said bore.

3. The tool of claim 1 wherein said release mechanism comprises a lever body pivotable about a lever axis, said lever axis being disposed substantially perpendicular to said first axis.

4. The tool of claim 1 wherein said first gripping member comprises an elongate shaft defining said first axis and said second member comprises a sleeve defining a bore, said shaft relatively moveably disposed within said bore, a distal end of said sleeve defining an opening to said bore, said gripping end extending outwardly from said opening, said bearing surface disposed on said distal end proximate said opening and said release mechanism further comprising a lever body pivotable about a lever axis, said lever axis being disposed substantially perpendicular to said first axis.

5. The tool of claim 4 wherein said gripping end is rotationally engageable with the internal surface and said first gripping member further comprises a radially projecting protrusion disposed at an end of said shaft opposite said gripping end, said protrusion being at least partially exposed.

6. A tool for releasably gripping an orthopedic implant having a threaded cavity, said tool comprising:
a first gripping member, said first gripping member having a threaded end threadingly engageable with the threaded cavity by relative rotation of said first gripping member and the implant about a first axis, said first gripping member postionable in at least one gripping position relative to the implant wherein said threaded end is threadingly engaged with the threaded cavity and is rotatable in both directions from said gripping position about said first axis relative to the implant;
a second gripping member having a bearing surface wherein, with said first gripping member in said gripping position, said second gripping member is translatable relative to said first gripping member in a direction substantially parallel to said first axis between an engaged position wherein said bearing surface is bearingly engaged with the implant proximate the threaded cavity and a disengaged position wherein said bearing surface is spaced from the implant; and
a release mechanism operable to release said first gripping member from a fixed axial position relative to said second gripping member defined by said engaged position of said second gripping member, said release mechanism being non-rotatable about said first axis.

7. The tool of claim 6 wherein said first gripping member comprises an elongate shaft and a radially projecting protrusion and said second gripping member comprises a sleeve defining a bore, said shaft being rotatably disposed in said bore, said threaded end extending outwardly from one end of said bore and said protrusion being at least partially exposed at an opposite end of said bore.

8. The tool of claim 6 wherein said release mechanism comprises a lever body pivotable about a lever axis, said lever axis being disposed substantially perpendicular to said first axis.

9. The tool of claim 6 wherein said release mechanism comprises a lever body pivotable about a lever axis, said lever axis being disposed substantially perpendicular to said first axis, said first gripping member comprises an elongate shaft and said second member comprises a sleeve defining a bore, said shaft rotatably disposed within said bore, wherein one end of said sleeve defines a distal end of said tool, said distal end defining an opening to said bore, said threaded end extending outwardly from said opening and said bearing surface disposed on said distal end proximate said opening.

10. The tool of claim 9 wherein said first gripping member further comprises a radially projecting protrusion disposed at an end of said shaft opposite said threaded end, said protrusion being at least partially exposed.

11. A tool for releasably gripping an orthopedic implant having an internally threaded cavity, said tool comprising:
a first gripping member having a shaft with a threaded end threadingly engageable with the cavity;
a tool body having a sleeve defining a bore extending through said sleeve, one end of said sleeve defining a distal end of said tool, said distal end defining an opening to said bore and a bearing surface proximate said opening wherein said shaft is rotatably disposed within said bore and extends through said opening to expose at least a portion of said threaded end;
a lever body pivotable about a lever axis, said lever axis oriented substantially perpendicular to said first axis, said lever body engageable with said first gripping member and said tool body, pivotal movement of said lever body axially displacing said first gripping member relative to said tool body, said lever body pivotally moveable between a first lever position wherein said threaded end extends a first distance outwardly from said sleeve opening to a second position wherein said threaded end extends a second distance outwardly from said sleeve opening, said first distance being greater than said second distance, said lever body operable to release said first gripping member from an axial position relative to said tool body defined by said lever body being in said second lever position with said threaded end and said bearing surface engaged with the implant.

12. The tool of claim 11 wherein said first gripping member further comprises a radially projecting protrusion disposed on an end of said shaft opposite said threaded end, said protrusion being at least partially exposed, and wherein said lever body is positioned between said threaded end and said protrusion and is engageable with said protrusion.

13. The tool of claim 11 wherein said distal end further comprises a feature rotationally engageable with the implant whereby engagement of said feature and the implant resists relative rotational movement of said tool and the implant.

14. The tool of claim 11 wherein said lever body is pivotally attached to said tool body and comprises a camming surface and a securement surface, said camming surface engageable with said first gripping member as said lever body is pivoted between said first lever position and said second lever position, said securement surface engaged with said first gripping member when said lever body is in said second lever position.

15. The tool of claim 14 wherein said securement surface is oriented substantially transverse to said shaft axis and wherein a line parallel to said first axis extending through said lever axis intersects said securement surface when said lever body is in said second lever position.

16. The tool of claim 14 wherein said securement surface is spaced from said lever axis by a distance which is no less than a first pivot distance, a first portion of said securement surface being spaced from said lever axis by said first pivot distance, said camming surface defining an arcuate surface separated from said lever axis by a variable distance no greater than said first pivot distance, a first portion of said camming surface being spaced from said lever axis by said first pivot distance, said first portion of said securement surface positioned adjacent said first portion of said camming surface.
